(19) United States
(12) Patent Application Publication
(10) Pub. No.: US 2025/0123265 A1
(43) Pub. Date: Apr. 17, 2025

(54) DIAPER RASH PREVENTION SYSTEM UTILIZING GAS ANALYSIS

(71) Applicant: Nozy Technologies Inc., Westlake Hills, TX (US)

(72) Inventors: Rabya Gillani, Westlake Hills, TX (US); Mark Lyle, Hernando Beach, FL (US)

(21) Appl. No.: 18/911,638

(22) Filed: Oct. 10, 2024

Related U.S. Application Data
(60) Provisional application No. 63/590,292, filed on Oct. 13, 2023.

(51) Int. Cl. G01N 33/497

(52) U.S. Cl. CPC G01N 33/497; G01N 2800/20

(57) ABSTRACT

A diaper rash prevention system includes an Internet of Things gas detection device equipped with at least one sensor that wirelessly communicates its readings to a communication device in the form of a software application running on a smartphone. The sensors inside the Internet of Things gas detection device analyze the gases in the air to determine when a child has a dirty diaper or underpants providing accurate and real-time notifications to caregivers.

### CROSS-REFERENCE TO RELATED APPLICATIONS

This application claims the benefit of U.S. Provisional Patent Application Ser. No. 63/590,292, entitled “DIAPER RASH PREVENTION SYSTEM UTILIZING GAS ANALYSIS,” filed October 13, 2023, which is incorporated herein by reference.

### BACKGROUND OF THE INVENTION

### 1. Field of the Invention

The present invention relates to a diaper rash prevention device.

### 2. Description of the Related Art

Diaper rash is a common problem faced by infants, babies, and/or toddlers (referred to below as “child” or “children”) caused by prolonged exposure to a dirty diaper. Prompt diaper changing is crucial to prevent diaper rash and maintain the child's comfort and hygiene. Existing solutions have limitations in accurately determining the state of a child's diaper, often relying on visual or tactile cues.

The present invention provides an innovative system that utilizes a network of sensors to analyze the gases in the air surrounding the child's crib, providing a reliable and timely indication of a dirty diaper.

### SUMMARY OF THE INVENTION

In one aspect a diaper rash prevention system includes an Internet of Things gas detection device equipped with at least one sensor that wirelessly communicates its readings to a communication device in the form of a software application running on a smartphone. The sensors inside the Internet of Things gas detection device analyze the gases in the air to determine when a child has a dirty diaper or underpants providing accurate and real-time notifications to caregivers.

In some embodiments the diaper rash prevention system the Internet of Things gas detection device is plugged into a wall outlet near a child's crib.

In some embodiments the diaper rash prevention system the Internet of Things gas detection device includes a housing supporting an electrical plug for attachment to an external power source.

In some embodiments the diaper rash prevention system the housing includes a cover and a base.

In some embodiments the diaper rash prevention system the cover is connected to the base for selective attachment thereto.

In some embodiments the diaper rash prevention system the cover is provided with a perforated grill allowing gases to readily pass therethrough such that they may be sensed by the sensors within the IoT gas detection device.

In some embodiments the diaper rash prevention system the electric plug is mounted along a base wall of the base of the housing.

In some embodiments the diaper rash prevention system the at least one sensor includes a network of sensors.

In some embodiments the diaper rash prevention system the network of sensors includes a CH4 (methane) sensor, a VOC (volatile organic compound) sensor, and a H2S (hydrogen sulfide) sensor.

In some embodiments the diaper rash prevention system the network of sensors is mounted on a circuit board.

In some embodiments the diaper rash prevention system the circuit board includes a processor, a transceiver, and a power source connection.

In some embodiments the diaper rash prevention system the Internet of Things gas detection device includes a fan.

In some embodiments the diaper rash prevention system the fan is positioned within a housing of the Internet of Things gas detection device such that it accesses the air that it needs to move, and the fan is positioned slightly above the at least one sensors such that the air being drawn in by the fan passes over and around the at least one sensors.

In some embodiments the diaper rash prevention system the diaper rash prevention system utilizes Artificial Intelligence and Machine Learning algorithms to process data collected from the at least one sensors to correlate and determine when a soiled diaper event has taken place.

Other objects and advantages of the present invention will become apparent from the following detailed description when viewed in conjunction with the accompanying drawings, which set forth certain embodiments of the invention.

### BRIEF DESCRIPTION OF DRAWINGS

FIG. 1 is a perspective view of the Internet of Things (IoT) gas detection device in use.

FIG. 2 is a schematic of the diaper rash prevention system.

FIG. 3 is an exploded view of the IoT gas detection device.

### DETAILED DESCRIPTION OF THE INVENTION

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

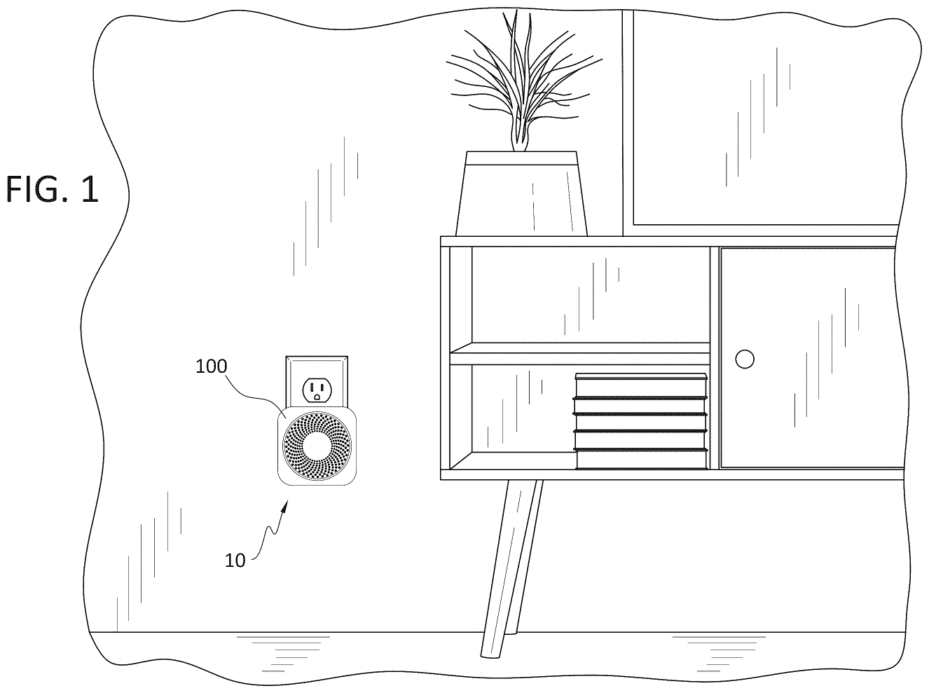

Referring to FIGS. 1 to 3, a diaper rash prevention system 10 is disclosed. The diaper rash prevention system 10 utilizes gas analysis in assisting caregivers as they take care of children, especially new parents.

The diaper rash prevention system 10 comprises an Internet of Things (IoT) gas detection device 100 equipped with a network of sensors 102, 104, 106. The IoT gas detection device 100 is plugged into a wall outlet near the child's crib and wirelessly communicates its readings to a communication device 200 in the form of a software application 202 running on a smartphone 204. The software application 202 is designed to provide real-time notifications of diaper soiling events and then track this activity over time by logging the diaper soiling events to look for and inform the caregiver of any detected patterns The sensors 102, 104, 106 inside the IoT gas detection device 100 analyze the gases in the air to determine when a child has a dirty diaper (or underpants). By providing accurate and real-time notifications to caregivers, the diaper rash prevention system 10 aims to prevent diaper rash in children and enhance overall childcare.

In accordance with a disclosed embodiment, the software application 202 provides a graphical user interface 206 displayed on a smartphone 204, or other computer device as is known in the art. The graphical user interface 206 includes main screen 208 with a dominant visual providing status information, settings information, sensor reading information, etc. as may be deemed necessary for optimal use of the diaper rash prevention system.

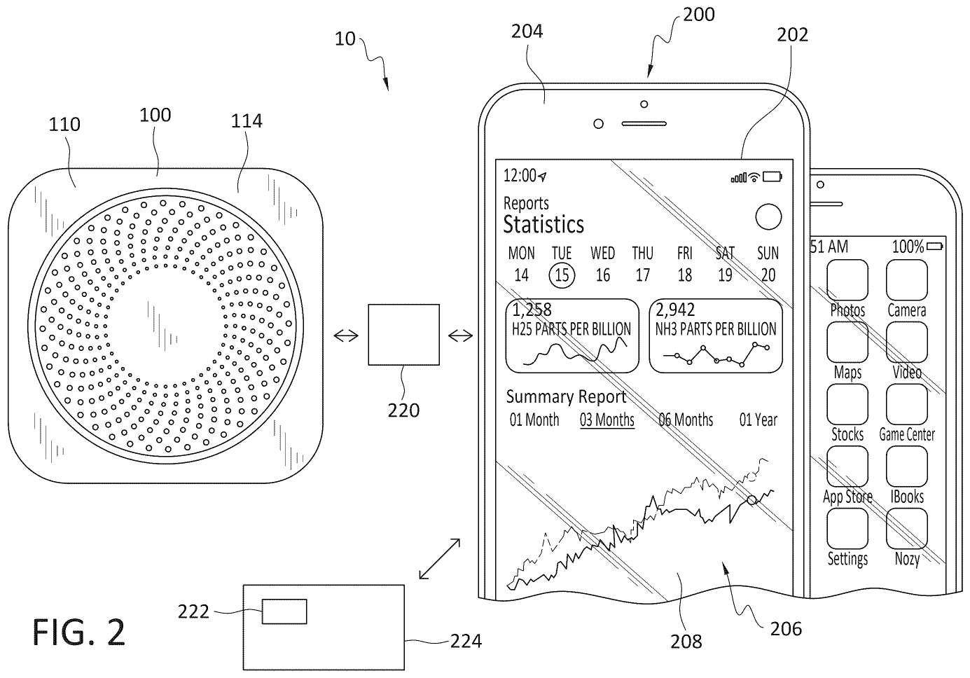

For example, the graphical user interface might include a prominent diaper icon, perhaps changing color or animation based on the gas levels detected (e.g., green for clean, red for needs changing). The graphical user interface might also include an identifier of the Time Since Last Change which may be in the form of a clear timer displaying how long it's been since the last diaper change. The graphical user interface might also include an identifier of the Gas Level Indicator which may be in the form of a subtle graph or bar showing the current levels of the monitored gases (see, for example, the graphs shown in FIG. 2). The graphical user interface might also include an identifier of the Quick Action Buttons, for example, large, easily tappable buttons for “Change Diaper” and “Snooze Alert” (temporarily silence notifications).

The software application graphical user interface might also provide a History Screen showing a Timeline View (that is, a visual representation of diaper changes over time, potentially using a calendar or graph format); Filtering Options providing for the ability to filter by date range, time of day, or type of soiling (if distinguishable); and Insights providing a summaries of average diaper change frequency, peak times of day for changes, etc.

The software application graphical user interface might also provide an interface for establishing Settings including, but not limited to, Notification Preferences offering Granular control over notification types (sound, vibration, visual), volume, and snooze duration; Sensitivity Adjustment providing users with the ability to tweak the sensitivity of the gas detection, potentially with presets for different age groups or individual needs; Device Management allowing the user to remotely connect/disconnect to the gas detection device, check battery levels (if applicable), and troubleshoot; Account & Data settings providing users with the ability to set privacy settings, data sharing options, and potentially cloud backup of diaper change history.

In addition to the features discussed above, the software application might further provide other controls and features, including, but not limited to, real-time gas monitoring providing live updates of the gas levels detected, with clear visual cues for when action is needed. The software application might also provide predictive algorithms to estimate when a diaper will likely need changing based on the rate of gas level increase and customizable alerts offering users a choice of notification types and intensity, the ability to set quiet hours or specific times when alerts should be suppressed, options for escalating alerts (e.g., increasing volume or frequency if the first alert is ignored). The software application might also provide diaper change logging allowing for automatic recording of diaper changes triggered by the software application, manual logging options for changes initiated by the caregiver independently, the ability to add notes or details to each change (e.g., type of soiling, any skin irritation observed). The software application might also provide for data analysis and insights providing for the presentation of diaper change patterns over time, helping parents identify any trends or potential issues and potential integration with other health tracking apps or wearables to provide a more holistic view of the child's well-being.

As briefly mentioned above, the diaper rash prevention system 10 includes an IoT gas detection device 100 and a communication device 200. The IoT gas detection device 100 is a compact and aesthetically pleasing device designed to be installed on a wall near the child's crib. The IoT gas detection device 100 contains a network of sensors 102, 104, 106 positioned strategically to analyze the gases in the air surrounding the child's crib. The IoT gas detection device 100 is equipped with wireless connectivity, enabling seamless communication with the communication device 200.

In accordance with a disclosed embodiment, the communication device 200 is a smartphone 204 loaded with a software application 202 providing a graphical user interface 206 such that the user is promptly notified when a diaper change is needed. In accordance with a disclosed embodiment, a standard pairing process for the smartphone 204 and the IoT gas detection device 100 is employed. The software application 202 of the smartphone 204 connects with the IoT gas detection device 100 over Bluetooth. The software application 202 then requests a list of nearby Wi-Fi networks that the IoT gas detection device 100 “sees.” The user then selects the preferred Wi-Fi network 220 from the list provided by the IoT gas detection device 100 and supplies the network password which is communicated back to the IoT gas detection device 100 over Bluetooth, after which the IoT gas detection device 100 attempts to connect to the preferred Wi-Fi network 220. If successful, the connection is logged in to a cloud database and the devices are now paired.

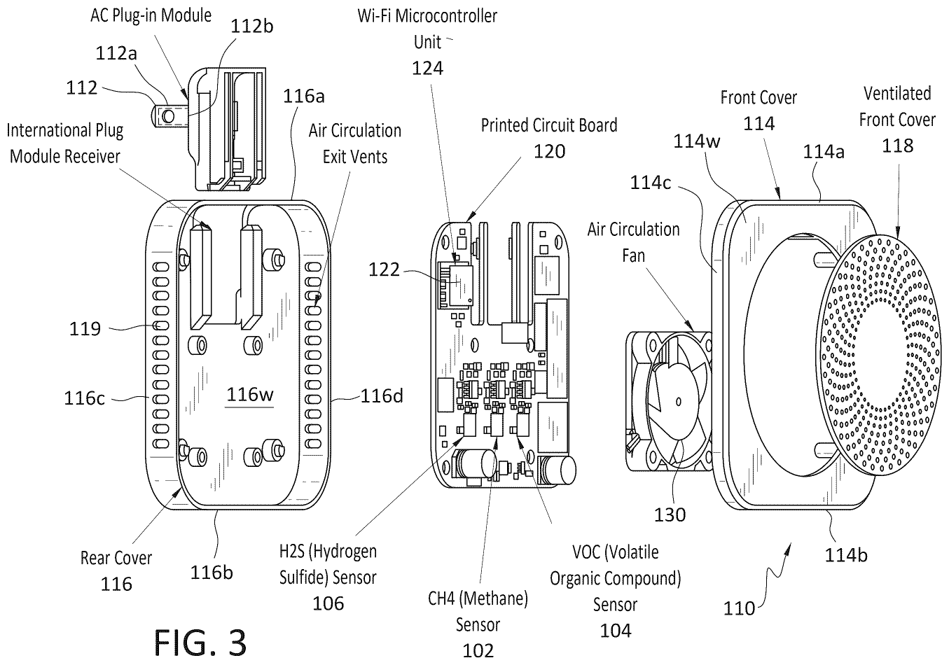

More specifically, and with reference to the IoT gas detection device 100 as shown in FIG. 3, it includes a network of sensors 102, 104, 106 designed to detect specific gases emitted from a dirty diaper, such as hydrogen sulfide and other volatile organic compounds. These sensors 102, 104, 106 are carefully calibrated to provide accurate and reliable readings, ensuring precise determination of diaper status. The sensor data is continuously monitored and analyzed to detect changes indicative of a dirty diaper.

The IoT gas detection device 100 includes a housing 110 supporting an electrical plug 112 for attachment to an external power source in the form of a conventional electrical outlet. The housing 110 is substantially rectangular in shape and includes a cover 114 and a base 116. The cover 114 is connected to the base 116 for selective attachment thereto. In accordance with a disclosed embodiment, the cover 114 is secured to the base 116 via a clamshell configuration with mating snap clip connections allowing secure attachment between the cover 114 and the base 116 without the need for specialized tools or coupling structures.

In accordance with a preferred embodiment, both the cover 114 and the base 116 include a concave construction such that when the housing 110 is closed the cover 114 and base 116 define an enclosed space. More particularly, the base 116 and cover 114 are connected such that users are not able to access the internal components thereof. The cover 114 is substantially rectangular in shape and includes first and second short side walls 114a, 114b and first and second long side walls 114c, 114d depending from the cover wall 114w, as well as an external surface and an interior surface on opposite sides of the cover 114. As will be appreciated based upon the following disclosure, the cover wall 114w is provided with a perforated grill 118 allowing gases to readily pass through the cover wall 114w such that they may be sensed by the sensors 102, 104, 106 within the IoT gas detection device 100.

The base 116 is substantially the same shape as the cover and is therefore rectangular in shape and includes first and second short side walls 116a, 116b and first and second long side walls 116c, 116d depending from the base wall 116w, as well as an external surface and an interior surface on opposite sides of the base 116. When in the closed configuration the short side walls 114a, 114b and long side walls 114c, 114d of the cover 114 align with the short side walls 116a, 116b and long side walls 116c, 116d of the base 116 with the respective external surfaces of the cover 114 and the base 116 facing away from each other. While a rectangular configuration is disclosed herein, it is appreciated the housing may be constructed with various shapes.

Considering the electric plug 112 used for attachment of the IoT gas detection device 100 to the external power source, the electric plug 112 is mounted along the base wall 116w of the base 116 of the housing 110. In accordance with a disclosed embodiment, the electric plug 112 is in the form of a module that is secured to the base 116. The electric plug module 112 may be permanently secured to the base 116 or it may be structured for selective attachment to the base 116 as is known in conjunction with other electronic devices. The electric plug module 112 includes standard first and second leg members 112a, 112b that are shaped and dimensioned for insertion within a conventional electric electrical outlet found within a wall or other structure. It is appreciated that the first and second leg members may be pivotally secured and adapted for rotational movement between their recessed first orientation and their extended second orientation.

While a disclosed embodiment includes a power source based upon on an electric plug inserted within an outlet, it is appreciated that other power systems may be implemented. For example, the IoT gas detection device could employ a battery (in particular, a rechargeable battery) allowing the IoT gas detection device to be positioned at optimal locations relative to the child's crib.

Within the housing 110 of the IoT gas detection device 100 are a number of functional elements allowing for operation in accordance with present invention. As briefly mentioned above, at least one sensor 102, 104, 106 is positioned within the housing 110. The sensor 102, 104, 106 is mounted on a circuit board 120 including a processor 122, a transceiver 124, and a power source connection 126. The circuit board 120 is further provided with additional common electronic components such as voltage regulators, operational amplifiers, etc. (not shown). While a disclosed embodiment is presented above, it is appreciated both individual discrete analog sensors and digitizing the outputs for analysis may be employed within the spirit of the present invention, In addition, it is appreciated an all-in-one digital sensor chipset might be used in accordance with the present invention.

Human feces range from 55% to 75% water. Much of the 25% to 45% that remains consists of gaseous methane—produced by bacterial breakdown. As such, and in accordance with a disclosed embodiment, the at least one sensor includes a CH4 (methane) sensor 102 capable of identifying methane found in feces associated with the bowel movements of children, a VOC (volatile organic compound), sensor 104 capable of identifying VOCs found in feces associated with bowel movements of children, and a H2S (hydrogen sulfide) sensor 106 found in feces associated with bowel movements of children. The sensors 102, 104, 106 are “off-the-shelf” items commonly available and utilized in smoke or carbon monoxide detectors. In accordance with a disclosed embodiment, they are metal oxide semiconductor (MOS) sensors that detect concentration of various types of gases by measuring the resistance change of the metal oxide due to adsorption of gases. While a CH4 (methane) sensor, VOC (volatile organic compound) sensor, and H2S (hydrogen sulfide) sensor are used in accordance with a disclosed embodiment, it is appreciated the type and number of sensors may be varied as the technology associated with the diaper rash prevention system advances. The transceiver 124 of the circuit board is a Wi-Fi microcontroller unit, although it is appreciated various communication devices and protocols such that the at least one sensor may communicate with the software application running on the smartphone.

While the disclosed embodiment is not intended to monitor urine, it is appreciated that human urine is composed primarily of water (95%). The rest is urea (2%), creatinine (0.1%), uric acid (0.03%), chloride, sodium, potassium, sulphate, ammonium, phosphate and other ions and molecules in lesser amounts. Humans release ammonia in urine. As such, it is likely that urine is susceptible to monitoring in accordance with the present invention.

In an effort to enhance air flow through the IoT gas detection device 100, the IoT gas detection device 100 includes a fan 130 positioned between the circuit board 120 and the grill 118 of the cover 114. The fan 130 is positioned within the housing 110 such that it accesses the air that it needs to move. In an effort to optimize the ability of the sensors 102, 104, 106 to identify gases, the fan 130 is positioned slightly above the sensors 102, 104, 106 such that the air being drawn in by the fan 130 passes over and around the sensors 102, 104, 106. However, it is appreciated that the relative position of the fan 130 and the sensors 102, 104, 106 may be varied to optimize operation of the IoT gas detection device 100. In addition, the side walls 116c, 116d of the base 116 are provided with air circulation vents 119. This arrangement allows air to be readily drawn into the housing 110, circulated about the CH4 (methane) sensor 102, the VOC (volatile organic compound) sensor 104, and the H2S (hydrogen sulfide) sensor 106, and exhausted through the air circulation vents 119.

The software application 202 is a user-friendly mobile application and operates on a smartphone 204 or other computer device. The software application 202 receives data wirelessly from the IoT gas detection device 100 and processes it to determine the diaper's cleanliness status. The software application 202 provides real-time notifications to caregivers, alerting them when a dirty diaper is detected.

In accordance with a disclosed embodiment, the diaper rash prevention system 10 utilizes Artificial Intelligence and Machine Learning algorithms 222 to process data collected from the sensors 102, 104, 106 to correlate and determine when a soiled diaper event has taken place. In accordance with a disclosed embodiment, the processing associated with the Artificial Intelligence and Machine Learning algorithms 222 is performed remotely from the software application 202. As such, the software application 202 is in contact with a central processing system 224 via the communication functionalities of the smartphone 204, which, in accordance with a disclosed embodiment, sends to and receives updates from the central processing system 224. The Artificial Intelligence and Machine Learning algorithms 202 apply the time stamped data received from the sensors 102, 104, 106 to optimize the provision of warnings to users. For example, it is known gas levels may vary through the day and night, the slope associated with the increase and/or decrease of gases is indicative of various events (for example, a steep upward slop with a gradual decline is commonly indicative of a bowel movement), and the positioning of IoT gas detection device 100 may have a bearing a gas levels used in the determination of warning events. This information and more is used by the Artificial Intelligence and Machine Learning algorithms 222 to update the systems understanding as to the likelihood that a warning is required and the urgency of such warnings. In summary, the data is collected on the device, then transmitted to the cloud (AWS) for analysis. Alerts will be triggered from the cloud to the software application (mobile app, smartwatch, etc). In the future, it is appreciated that some of this processing may be moved to the IoT device.

Additionally, and as discussed above, the software application 202 offers customization options allowing users to set preferences for notifications and access historical diaper change records such that caregivers are provided with real-time notifications and records identifying soiled diapers/diaper changes.

In practice, air is continuously drawn into the housing 110 of the IoT gas detection device 100 by the fan 130. The air drawn into the housing 110 is circulated about the CH4 (methane) sensor 102, the VOC (volatile organic compound) sensor 104, and the H2S (hydrogen sulfide) sensor 106. The CH4 (methane) sensor 102, the VOC (volatile organic compound) sensor 104, and the H2S (hydrogen sulfide) sensor 106 generate raw electrical signals based upon the sensed air and transmit these raw signals to the software application 202. Considering that the flow of air through the housing 110 is important to ensure the sensors 102, 104, 106 are exposed to levels of gases necessary for identification of events at issue, the positioning of the housing relative to a crib or bed is important. In accordance with a disclosed embodiment, simply plugging the IoT gas detection device 100 into a wall outlet (at a height of approximately 16 inches as dictated by common construction guidelines) adjacent the crib or bed is sufficient to ensure that the IoT gas detection device 100 is exposed to adequate levels of gas for making determinations regarding how and when warnings are issued.

The transmission of the raw signals is facilitated by the Wi-Fi microcontroller unit 124 operating in conjunction with a household Wi-Fi network 220 to ensure the raw data is promptly transmitted and delivered to the software application 202 operating on the smartphone 204.

The software application 202 operating on the smartphone 204 monitors the raw signals to identify changes. Upon the identification of a change indicating a possible need for a diaper change, a warning (audio, visual, or both) is generated by the software application 202 operating on the smartphone 204 to inform the caregiver that a diaper change is likely needed.

While the present system is disclosed above for use in conjunction with newborns, babies, infants, and toddlers, it is appreciated the system may be implemented in a variety of environment where prompt identification of body waste is important to maintaining the health of those people being taken care of. That is, the present system can be used across all age groups, covering various environments that require health monitoring or hygiene management. For example, it is appreciated that the system would be highly beneficial to those operating and living in nursing homes.

While the preferred embodiments have been shown and described, it will be understood that there is no intent to limit the invention by such disclosure, but rather, is intended to cover all modifications and alternate constructions falling within the spirit and scope of the invention.

1. A diaper rash prevention system, comprising:
an Internet of Things gas detection device equipped with at least one sensor that wirelessly communicates its readings to a communication device in the form of a software application running on a smartphone;
wherein the sensors inside the Internet of Things gas detection device analyze the gases in the air to determine when a child has a dirty diaper or underpants providing accurate and real-time notifications to caregivers.

2. The diaper rash prevention system according to claim 1, wherein the Internet of Things gas detection device is plugged into a wall outlet near a child's crib.

3. The diaper rash prevention system according to claim 1, wherein the Internet of Things gas detection device includes a housing supporting an electrical plug for attachment to an external power source.

4. The diaper rash prevention system according to claim 3, wherein the housing includes a cover and a base.

5. The diaper rash prevention system according to claim 4, wherein the cover is connected to the base for selective attachment thereto.

6. The diaper rash prevention system according to claim 4, wherein the cover is provided with a perforated grill allowing gases to readily pass therethrough such that they may be sensed by the sensors within the IoT gas detection device.

7. The diaper rash prevention system according to claim 4, wherein the electric plug is mounted along a base wall of the base of the housing.

8. The diaper rash prevention system according to claim 1, wherein the at least one sensor includes a network of sensors.

9. The diaper rash prevention system according to claim 8, wherein the network of sensors includes a CH₄ (methane) sensor, a VOC (volatile organic compound) sensor, and a H₂S (hydrogen sulfide) sensor.

10. The diaper rash prevention system according to claim 8, wherein the network of sensors is mounted on a circuit board.

11. The diaper rash prevention system according to claim 10, wherein the circuit board includes a processor, a transceiver, and a power source connection.

12. The diaper rash prevention system according to claim 1, wherein the Internet of Things gas detection device includes a fan.

13. The diaper rash prevention system according to claim 12, wherein the fan is positioned within a housing of the Internet of Things gas detection device such that it accesses the air that it needs to move, and the fan is positioned slightly above the at least one sensors such that the air being drawn in by the fan passes over and around the at least one sensors.

14. The diaper rash prevention system according to claim 1, wherein the diaper rash prevention system utilizes Artificial Intelligence and Machine Learning algorithms to process data collected from the at least one sensors to correlate and determine when a soiled diaper event has taken place.
